(19) United States
(12) Patent Application Publication
(10) Pub. No.: US 2022/0409531 A1
(43) Pub. Date: Dec. 29, 2022

(54) PHARMACEUTICAL DELIVERY DEVICE AND METHOD OF MANUFACTURE

(71) Applicant: University of Ulster, Coleraine, County Londonderry (GB)

(72) Inventors: Ming-Wei Chang, Newtownabbey, County Antrim (GB); James McLaughlin, Belfast, County Antrim (GB)

(21) Appl. No.: 17/764,037

(22) PCT Filed: Sep. 24, 2020
(86) PCT No.: PCT/EP2020/076768

§ 371 Date: Mar. 25, 2022

(30) Foreign Application Priority Data
Sep. 27, 2019 (GB) 1913972.4

(51) Int. Cl. A61K 9/00; D01D 5/00; A61K 9/51

(52) U.S. Cl. CPC A61K 9/0092; D01D 5/003; A61K 9/5192

(57) ABSTRACT

A pharmaceutical delivery device, comprising a cylindrical body formed from a plurality of concentrically arranged layers, each layer being formed from a biodegradable material and incorporating at least one active pharmaceutical agent. Optionally, the device comprises an outer layer, and inner layer and one or more intermediate layers, wherein at least one of the one or more intermediate layers is formed from a material having a greater rate of degradation that the inner and outer layers such that the inner and outer layers separate in use.

### CROSS REFERENCE TO RELATED APPLICATIONS

The present application is a § 371 national stage of International Application PCT/EP2020/076768, filed Sep. 24, 2020, which claims priority benefit to U.K. Pat. Application Ser. No. 1913972.4, filed Sep. 27, 2019, both of which are hereby incorporated herein by reference in their entireties.

### FIELD OF THE INVENTION

This invention relates to an improved pharmaceutical delivery device and method of manufacture thereof and in particular to a pharmaceutical delivery device and method of manufacture that can be customized according to a patient's needs.

### BACKGROUND OF THE INVENTION

Personalised medicine is a medical model that proposes the customization of healthcare, with medical decisions, practices, and/or products being tailored to the individual patient. A specific pharmaceutical delivery device is selected and crafted for an individual patient based on various properties (e.g., age, ingredient selection, dose level, route of administration, etc.). Traditionally, such customised pharmaceutical delivery devices comprise solid capsules in which the pharmaceutical is enclosed within a soluble shell with diameters ranging from 5.0 mm to 8.1 mm, and a capsule length between 12.1 mm and 19.8 mm. Capsules have the advantages that they are easily administered, are able to cover up unpleasant odours and tastes, and can dissolve rapidly. Oral formulations still remain the most widely used pharmaceutical delivery mechanism when dealing with treatment of chronic diseases and cancer therapy and have led to a paradigm shift from single-pharmaceutical-to-one-target approaches to the use of combinations of multi-target pharmaceuticals to synergistically overcome several therapeutic challenges clinicians facing today. However, methods of producing capsules containing multiple pharmaceuticals and having specific release profiles that are truly customizable are limited, challenging, and expensive. Accordingly, there is a significant unmet need for a method of producing fully customizable pharmaceutical delivery devices.

In the manufacture of orally administered pharmaceuticals, a range of dosage forms are used to enclose medicines in a relatively stable shell for oral delivery known as a capsule. In 1847, James Murdock of London patented the two-piece telescoping gelatin capsule by dipping metal pins in the gelling agent solution, (IOSR Journal of Dental and Medical Sciences (IOSR-JDMS) e-ISSN: 2279-0853, p-ISSN: 2279-0861. Volume 15, Issue 1 Ver. VII (January 2016), PP 41-49, www.iosrjournals.org). Dry powder and other dosage forms such as beads, tablets, and even oils can be filled into the capsule shell. From the patient perspective, the capsules have many advantages such as odourless, tasteless, elegant, easy-to-swallow, and easy-to-fill shell, making them among the most popular pharmaceutical delivery devices on the market. Currently, methods of producing a capsule shell with release profiles and real time imaging capabilities that is truly customizable are limited, challenging, and expensive. Accordingly, there is a significant unmet need for a method of producing fully customizable pharmaceutical delivery devices.

There is need in the art for providing pharmaceutical oral dosage formulations that can be personalized in dosage while providing the desired release characteristics, in particular controlled release for immediate, delayed, or sustained release (examples are disclosed in WO2001087272A2 WO2003092633A2, WO2013112882A1, WO2017004122A1). However, the high temperature of printing nozzles during operation of known 3D printing techniques is not compatible with many thermosensitive pharmaceutical compounds. Also, precision control of the millimetre-scale-diameter filament used in FDM 3D printing remains challenging in terms of accurate dosage control and the resulting pharmaceutical release behaviours. It also should be noted that it is hard to encapsulate polypharmaceuticals in a single oral capsule with contrast agents for clinical imaging. There is further a need for a method of producing pharmaceutical capsules using polymers which have higher controllable resolution, in particular, i.e. controlled-release rates, function properties, polypharmaceutical and from which oral dosage formulations can be printed.

### SUMMARY OF THE INVENTION

According to a first aspect of the present invention there is provided a pharmaceutical delivery device, comprising a cylindrical body formed from a plurality of concentrically arranged layers, each layer being formed from a biodegradable material and incorporating at least one active pharmaceutical agent.

In one embodiment the device comprises an outer layer, and inner layer and one or more intermediate layers, wherein at least one of the one or more intermediate layers is formed from a material having a greater rate of degradation that the inner and outer layers such that the inner and outer layers separate in use.

The inner layer may have a first cylindrical geometric shape, the at least one intermediate layer covering the inner layer, the outer layer covering the inner and at least one intermediate layers.

In another embodiment the layers may be concentrically asymmetric to one another.

The inner and outer layers may comprise one of polylactic acid (PLA), poly-ε-caprolactone (PCL) or cellulose acetate (CA) and the at least one intermediate layer may comprise one of polyvinylpyrrolidone (PVP) or polyethylene glycol (PEG).

The inner and outer layers may comprise aligned fibres, the at least one intermediate layer comprising fibres deposited in a random orientation.

At least one layer incorporates ferromagnetic nanoparticles to enable the layer to be susceptible to movement under the effect of an external magnetic field.

In yet another embodiment at least one layer may incorporate a contrast agent having Fe3O4 nanoparticles for T1 and T2 response using an MRI imaging system.

According to a further aspect of the present invention there is provided a method of delivering a pharmaceutical delivery device as claimed in any preceding claim, comprising administering the pharmaceutical delivery device to a subject, wherein, upon contact with a surrounding solvent, at least one intermediate layer of the device is dissolved more rapidly than outer and inner layers of the device to deliver fast pharmaceutical release, the inner and outer layers separating upon dissolution of the at least one intermediate layer to separately dissolve at predetermined rates with continuous pharmaceutical release.

According to a further aspect of the present invention there is provided a method of producing a pharmaceutical delivery device, comprising:

a) electrohydrodynamic printing a first solution onto a cylindrical collector, comprising the first polymer and at least one active pharmaceutical agent, to form an inner layer of the device;
b) electrospinning a second solution comprising a second polymer and at least one active pharmaceutical agent to form a middle layer on the inner layer;
c) electrohydrodynamic printing a third solution comprising a third polymer and at least one active pharmaceutical agent to form an outer layer on the middle layer;
d) removing the complete cylindrical delivery device from the cylindrical collector.

The active pharmaceutical agent and layer thickness of each layer of the device may be determined according to a diagnosis made for a patient in need of treatment.

These and other objects, advantages and features of the invention will become apparent upon review of the following specification in conjunction with the drawings.

### BRIEF DESCRIPTION OF THE DRAWINGS

FIG. 1 illustrates a pharmaceutical delivery device in accordance with an embodiment of the present invention;

FIG. 2 shows detailed schematics illustrating the method of manufacture of the pharmaceutical delivery device of FIG. 1;

FIG. 3 illustrates an experimental image of the pharmaceutical delivery device of FIG. 1;

FIG. 4 illustrates an experimental demonstration of the use of the pharmaceutical delivery device of FIG. 1 illustrating the separation of the inner and outer layers of the device following preferential dissolution of the intermediate layer;

FIG. 5 shows illustrates the capability of the outer layer of a pharmaceutical delivery device in accordance with an embodiment of the present invention, containing nanoparticles, of targeting from left to right under an external magnetic field;

FIGS. 6A-6D illustrate an experimental demonstration of a pharmaceutical delivery device in accordance with an embodiment of the present invention with different Fe3O4 nanoparticles concentrations (1%, 2% and 3% of the PCL mass) showing that it is capable of MRI imaging in which FIGS. 6A and 6B are different views from T1 imaging, and FIGS. 6C and 6D are different views of T2 imaging;

FIGS. 7A and 7B illustrate the use of a pharmaceutical delivery device in accordance with an embodiment of the present invention to release two pharmaceuticals at the same time, each with their own unique release profile, in which FIG. 7A is a pharmaceutical release profile of APAP and CM from the various capsule configurations in a simulated gastric fluid (pH=1.7) and a simulated intestinal fluid (pH=7.4), and FIG. 7B is a detailed pharmaceutical release profile for APAP and CM from the composite capsule in the first two hours in a simulated gastric fluid (pH=1.7); and

FIG. 8 shows low cytotoxicity results with respect to each component of a pharmaceutical delivery device in accordance with an embodiment of the present invention.

### DETAILED DESCRIPTION OF THE PREFERRED EMBODIMENTS

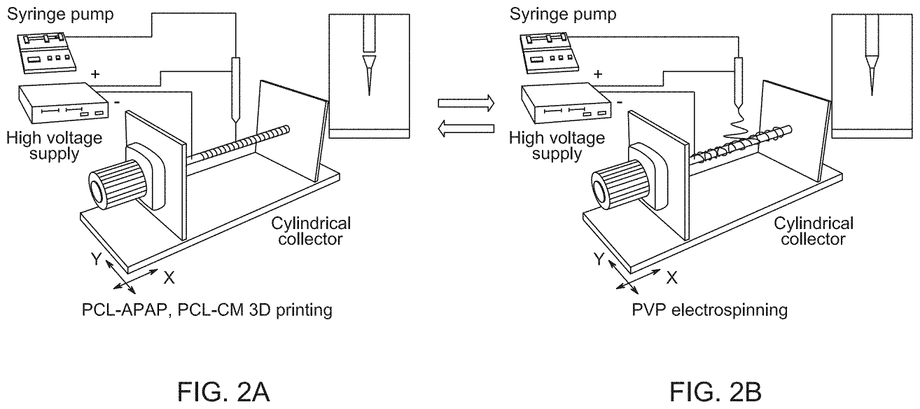

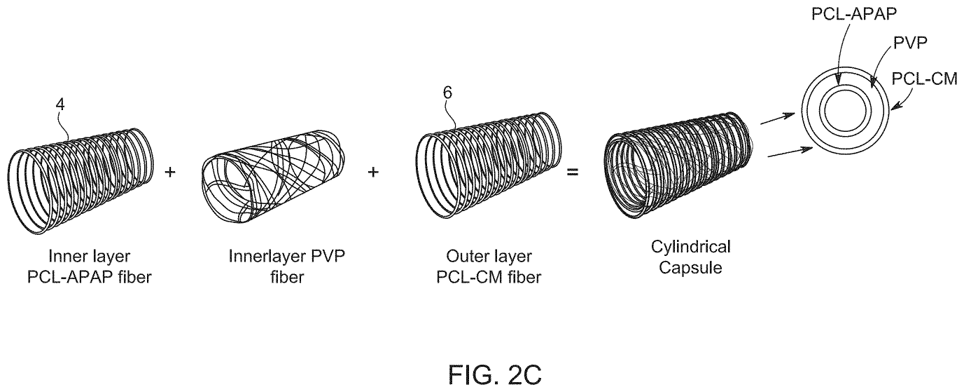

A pharmaceutical delivery device in accordance with an embodiment of the present invention comprises inner and outer PCL layers, which may be fabricated using EHD printing, and at least one electro-spun PVP intermediate layer, wherein the layers are combined into a composite concentric cylinder. The layers of the device define cylindrical concentric shells with each layer having a distinctive functional pharmaceutical component and a distinctive release profile. For example, poly ε-caprolactone (PCL) with paracetamol (APAP) and chlorpheniramine maleate (CM), synergistic pharmaceuticals commonly used to alleviate influenza symptoms, may be printed as an inner layer and outer layer respectively, via electrohydrodynamic (EHD) printing of micro-scaled fibres (see FIG. 2a).

Polyvinyl pyrrolidone (PVP) nanofibres may be embedded as one or more intermediate layers between the inner and outer PCL-pharmaceutical layers using an electrospinning (ES) techniques (see FIG. 2b). In one embodiment the resulting concentric cylindrical capsule may be produced to have a 6 mm diameter and 15 mm length to enable it to be swallowed for oral pharmaceutical delivery.

The present invention provides a straightforward fabrication method to customize mixtures of orally delivered pharmaceuticals with different solubilities and dosage profiles by innovatively using 3D printing tools and techniques that facilitate precision medicine and healthcare applications. After dissolution of the preferentially biodegradable PVP intermediate layer, the capsule separates in two parts, respectively comprising the inner and outer layers of the capsule, for separate and continuous pharmaceutical dosing.

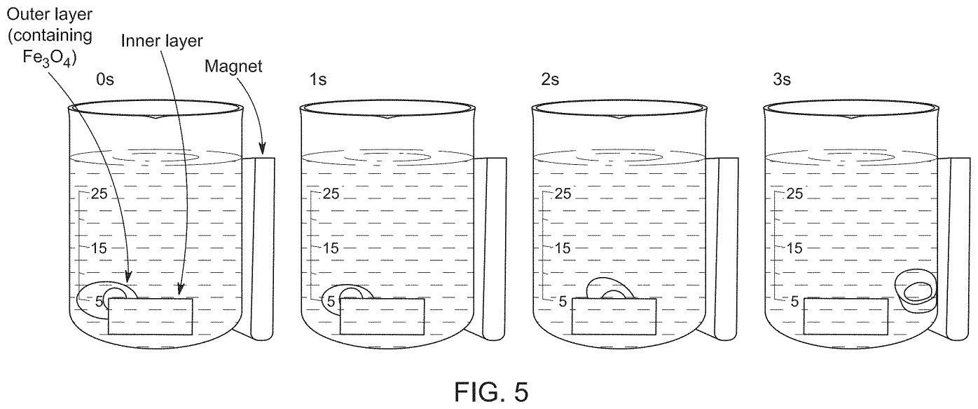

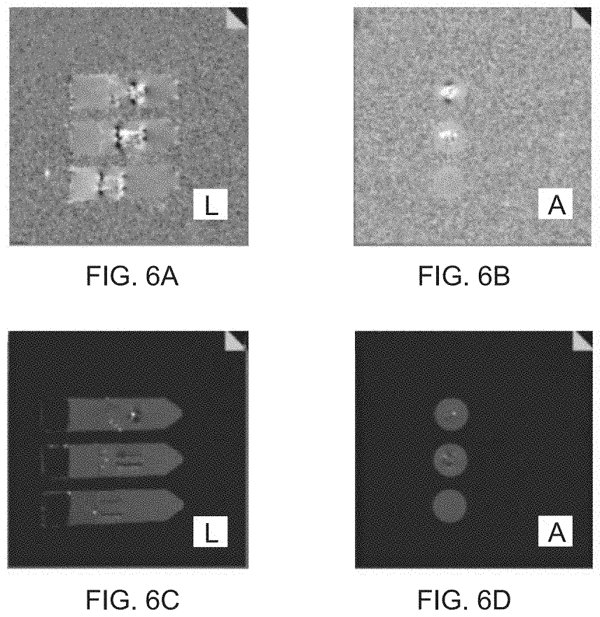

One or more layers of the capsule may incorporate tracer or contrast agents, such as Fe3O4 nanoparticles. As illustrated in FIG. 5, in vitro testing with gastric and intestinal fluids demonstrate the externally-directed targeting capabilities of such devices via the incorporation of such nanoparticles. Also, capsule imaging has been observed using a 3T MRI system which allowed temporal tracking of the capsule position (see FIG. 6).

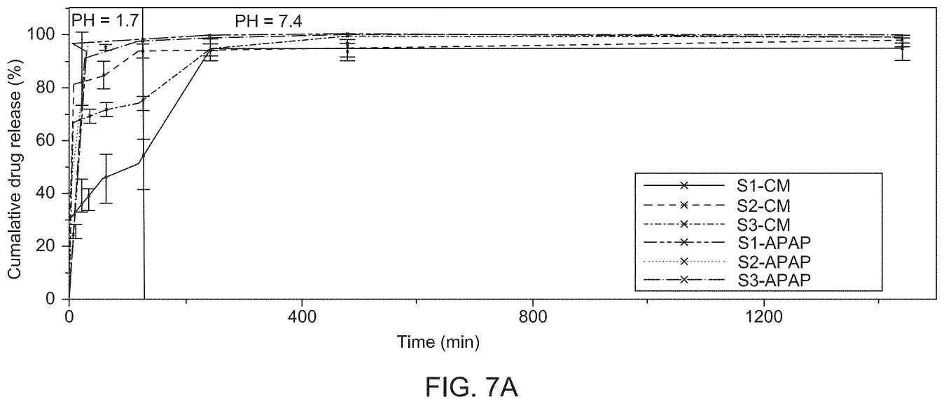

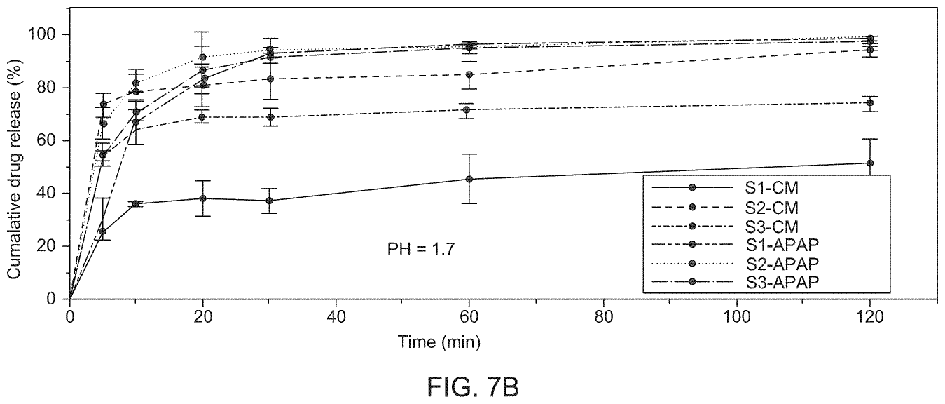

The release of two pharmaceuticals at the same time, each with their own unique release profile, can be controlled by selection of the polymer composite and layer thickness of each layer of the capsule (see FIG. 7). This provides several advantages since it uses micro and nano-scaled 3D printing techniques to demonstrate controlled pharmaceutical dosing, multi-pharmaceutical delivery, and integrated pharmaceutical tracking/imaging for testing of emerging combinatorial treatments in personalized medicine based on existing pharmaceuticals with customized release profiles and targeted delivery via the use of ferromagnetic nanoparticles that can be susceptible to magnetic fields.

Oral pharmaceutical delivery is the preferred route for pharmaceutical administration due to its convenience, cost-effectiveness, and high patient compliance. The incorporation of novel tracer agents, such as ferromagnetic nanoparticles, can provide externally-directed targeting capabilities. This can arguably address a current bottleneck in personalized medicine which is the rapid development of custom therapeutic solutions based on existing pharmaceuticals with customized release profiles and targeted delivery.

The present invention provides a new type of capsule comprising of printed layers rolled into cylindrical concentric shells with each layer having a distinctive functional pharmaceutical component. Poly c-caprolactone (PCL) with paracetamol (APAP) and chlorpheniramine maleate (CM), synergistic pharmaceuticals commonly used to alleviate influenza symptoms, can be respectively printed as an inner layer and outer layer, via electrohydrodynamic (EHD) printing of micro-scaled fibres. Polyvinyl pyrrolidone (PVP) nanofibres may be embedded as one or more intermediate layers between the two printed PCL-pharmaceutical layers, such as by using electrospinning (ES) techniques. In use, after dissolution of the PVP intermediate layer or layers, the capsule separates in two, with inner and outer layers providing separate and continuous pharmaceutical dosing. In vitro testing with gastric and intestinal fluids demonstrated controllable separation times for both the outer and inner capsules. Imaging was achieved using a 3T MRI system which allowed temporal tracking of the capsule components though the incorporation of nanoparticles (Fe3O4).

Key advantages of embodiments of the present invention:—

New customized oral capsules for personalized medicine delivery for “single-capsule-multi-targets” via electrohydrodynamic (EHD) printing of micro-scaled fibres; High precision printing of cylindrical capsules with multifunctional layers;

Each layer can be used to encapsulate different pharmaceuticals/biomarkers with desirable pharmaceutical release/function;

After dissolution of the one or more intermediate layers, the capsule separates in two parts, respectively comprising the inner and outer layers, for separate and continuous pharmaceutical dosing;

Fe3O4 nanoparticles may be incorporated to the device, providing an opportunity for targeted capsule position and trace release of the separate cylinders via MRI imaging;

In vitro testing of the release of the two pharmaceuticals indicates that the release of APAP and CM from the fibres mostly fit the Higuchi model;

Pharmaceutical delivery devices in accordance with the present invention have been shown to be biocompatible based on tests with L929 cell cultures.

### EXAMPLES

Cylindrical Capsules

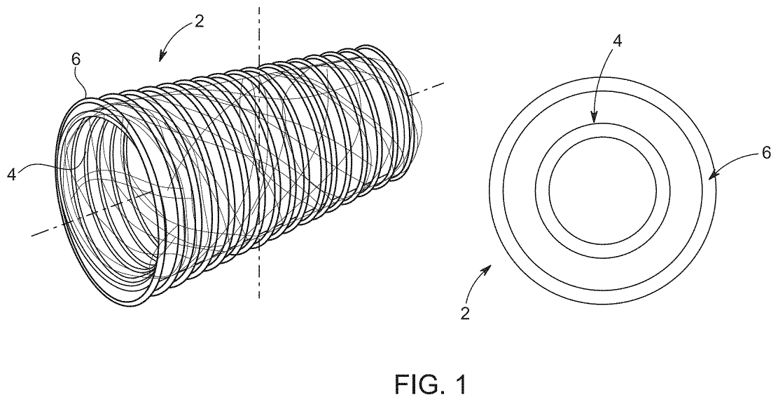

As illustrated in FIG. 1, a cylindrical capsule 2 in accordance with an embodiment of the present invention with multifunctional layers for personalized oral delivery has a 6 mm inner diameter and is 15 mm in length. The capsule has an inner layer 4 and an outer layer 6. Such capsule 2 may be fabricated by combining 3D printing and ES technology with a cylindrical collector. Both the inner layer 4 (PCL-APAP fibres) and outer layer 6 (PCL-CM fibres) of the capsule may be fabricated via EHD 3D printing using the same parameters. An EHD 3D printing system (see FIG. 2(a)), which includes a syringe pump, high voltage supply, X-Y motion translation platform and cylindrical collector, may be used to fabricated the inner layer and outer layers of the cylindrical capsule. During EHD 3D printing, the flow rate was set at 0.3 ml/h and the voltage was set at 2.5-2.7 kV. The distance between the metal needle and the cylindrical collector was set at 3-4 mm. The rotation speed of cylindrical collector was set at 150 rpm and the X-axis moved at 6 mm/s between two points whose distance was 15 mm apart and this was repeated 150 times. An intermediate layer (PVP layer) was fabricated via electrospinning (FIG. 2(b)) with the same equipment as used for EHD 3D printing at a flow rate of 0.8 ml/h for the PVP solution and at voltages from 8-10 kV. The distance between the metal needle was set at 10-15 mm and the cylindrical collector was rotating at 600 rpm. The fabrication of the complete cylindrical capsule, as illustrated in FIG. 2(c), has a PVP intermediate layer that separates the PCL-APAP and PCL-CM inner and outer layers.

Results

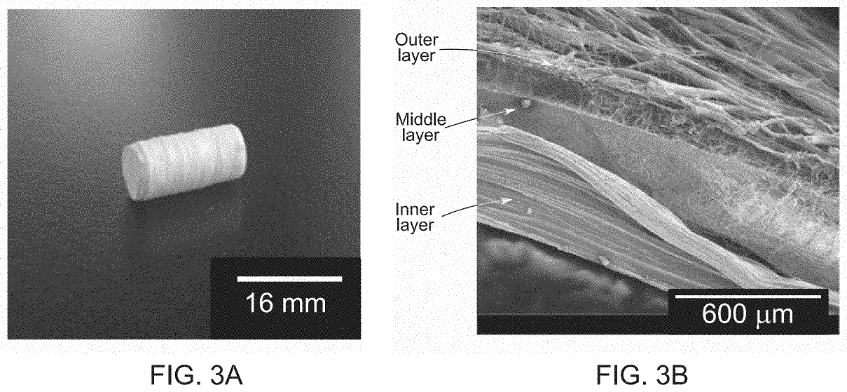

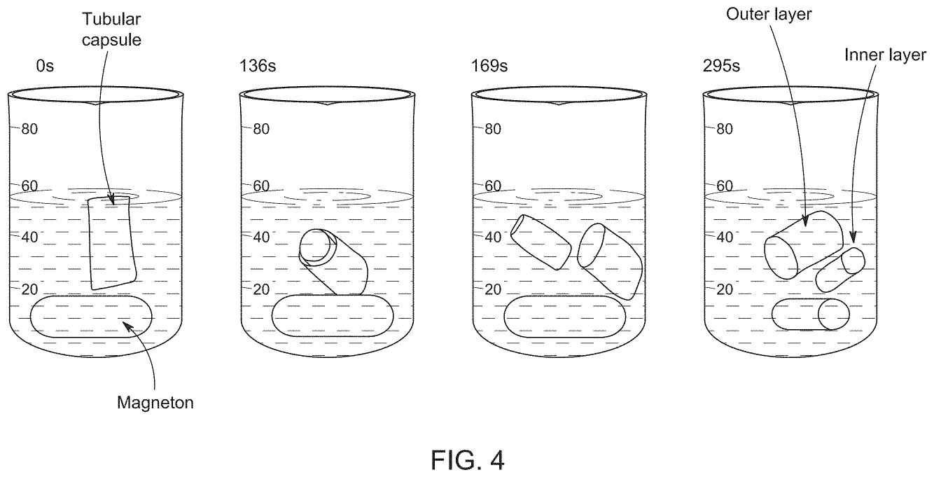

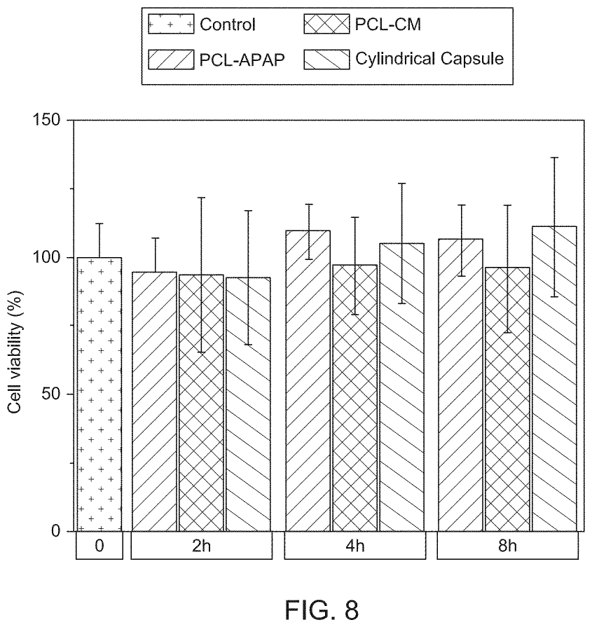

FIG. 3a shows the cylindrical capsule generated using EHDP and ES methods. FIG. 3b shows the integration process and a cross-sectional illustration of the complete capsule containing an inner layer of PCL-APAP fibres, a middle layer of PVP fibres and an outer layer of PCL-CM fibres. In addition, in order to demonstrate the versatility of capsule separation, in vitro testing was performed to observe separation of a composite capsule. FIG. 4 shows the separation process at different time intervals from 0 s to 295 s. The capsule sample was immersed into a 100 ml beaker containing 50 mL PBS solution being stirred at 100 rpm. The whole separation process was finished in 169 s and the complete dissolution of PVP finished until 295 s. Also, regulation and controlled movement of each component pharmaceutical layer after separation was achieved by adding Fe3O4 nanoparticles and an external magnetic field. The separated capsule containing the printed outer PCL with embedded Fe3O4 nanoparticles and travel quickly from left to right under an applied external magnet field while the inner layer without any nanoparticles remained stationary (FIG. 5). This travel distance was covered within 3 s at a velocity of 6.7 mm/s. In addition, FIG. 6 shows T1 graphing and T2 imaging using the 3T MRI system. This allows for multifunctional uses of the capsule such as for targeted pharmaceutical release using an externally located magnet, and as a magnetic resonance tracer. Pharmaceutical release profiles of APAP and CM known to be clinically important from the various capsule configurations in a simulated gastric fluid (pH=1.7) and a simulated intestinal fluid (pH=7.4) were demonstrated (FIG. 7a). The release of APAP was faster than CM during the first 2 hours in a simulated gastric fluid (pH=1.7) (FIG. 7b). The cumulative release of APAP was more than 90% while the cumulative release of CM was around 70%. Biocompatibility of the capsule and its components was demonstrated (FIG. 8). The results show cell proliferation after adding the capsule and its components into the L929 cells culture dish.

The invention is not limited to the embodiment described herein but can be amended or modified without departing from the scope of the present invention as defined by the appended claims as interpreted according to the principles of patent law including the doctrine of equivalents.

1. A pharmaceutical delivery device, comprising a cylindrical body formed from a plurality of concentrically arranged layers, each layer being formed from a biodegradable material and incorporating at least one active pharmaceutical agent.

2. The device of claim 1, comprising an outer layer, and inner layer, and one or more intermediate layers, wherein at least one of said one or more intermediate layers is formed from a material having a greater rate of degradation than said inner and outer layers such that said inner and outer layers separate in use.

3. The device of claim 2, wherein said inner layer has a first cylindrical geometric shape, said one or more intermediate layers covering said inner layer said outer layer covering said inner and at least one intermediate layers.

4. The device of claim 1, wherein said layers are concentrically asymmetric.

5. The device of claim 2, wherein said inner and outer layers comprise one of polylactic acid (PLA), poly-ε-caprolactone (PCL) or cellulose acetate (CA) and said one or more intermediate layers comprise one of polyvinylpyrrolidone (PVP) or polyethylene glycol (PEG).

6. The device of claim 2, wherein said inner and outer layers comprise aligned fibres, said one or more intermediate layers comprising fibres deposited in a random orientation.

7. The device of claim 1, wherein at least one of said layers incorporates ferromagnetic nanoparticles.

8. The device of claim 1, wherein at least one of said layers incorporates a contrast agent having Fe3O4 nanoparticles for T1 and T2 response using an MRI imaging system.

9. A method of delivering a pharmaceutical with a pharmaceutical delivery device comprising a cylindrical body formed from a plurality of concentrically arranged layers, each layer being formed from a biodegradable material and incorporating at least one active pharmaceutical agent, said method comprising administering the pharmaceutical delivery device to a subject, wherein, upon contact with a surrounding solvent, at least one intermediate layer of the device is dissolved more rapidly than outer and inner layers of the device to deliver fast pharmaceutical release, the inner and outer layers separating upon dissolution of the at least one intermediate layer to separately dissolve at predetermined rates with continuous pharmaceutical release.

10. A method of producing a pharmaceutical delivery device, said method comprising:
a) electrohydrodynamic printing a first solution onto a cylindrical collector, the cylindrical collector comprising a first polymer and at least one active pharmaceutical agent, to form an inner layer of the device;
b) electrospinning a second solution comprising a second polymer and at least one active pharmaceutical agent to form a middle layer on the inner layer;
c) electrohydrodynamic printing a third solution comprising a third polymer and at least one active pharmaceutical agent to form an outer layer on the middle layer and resulting in a complete cylindrical delivery device; and
d) removing the complete cylindrical delivery device from the cylindrical collector.

11. The method of claim 10, further comprising selecting the at least one active pharmaceutical agent and a thickness of each layer of the device according to a diagnosis made for a patient in need of treatment.
